SEQUENCE LISTING (14 sequences)

SEQ ID NO: 1 - protein, 11 aa, Artificial Sequence
  Synthesized
RRYIGRGYIRR

SEQ ID NO: 2 - protein, 9 aa, Artificial Sequence
  synthesized
RLYLRIGRR

SEQ ID NO: 3 - protein, 9 aa, Artificial Sequence
  synthesized
CRVRGGRCA

SEQ ID NO: 4 - protein, 6 aa, Artificial Sequence
  synthesized
RRRRRR

SEQ ID NO: 5 - protein, 22 aa, Artificial Sequence
  synthesized
RRRRRRGALAGRRRRRRGALAG

SEQ ID NO: 6 - protein, 23 aa, Artificial Sequence
  synthesized
GRRRRRRGALAGRRRRRRGALAG

SEQ ID NO: 7 - protein, 22 aa, Artificial Sequence
  synthesized
KKKKKKGALAGKKKKKKGALAG

SEQ ID NO: 8 - protein, 11 aa, Artificial Sequence
  synthesized
CRLYLRIGRRC

SEQ ID NO: 9 - protein, 24 aa, Artificial Sequence
  synthesized
CRRRRRRGALAGRRRRRRGALAGC

SEQ ID NO: 10 - protein, 25 aa, Artificial Sequence
  synthesized
CGRRRRRRGALAGRRRRRRGALAGC

SEQ ID NO: 11 - protein, 10 aa, Artificial Sequence
  artificial
CRVRGGRCAC

SEQ ID NO: 12 - protein, 8 aa, Artificial Sequence
  synthesized
CRRRRRRC

SEQ ID NO: 13 - protein, 24 aa, Artificial Sequence
  synthesized
CKKKKKKGALAGKKKKKKGALAGC

SEQ ID NO: 14 - protein, 13 aa, Artificial Sequence
  synthesized
CRRYIGRGYIRRC

(19) United States
(12) Patent Application Publication
(10) Pub. No.: US 2022/0033951 A1
(43) Pub. Date: Feb. 3, 2022

(54) Functionalized Surface

(71) Applicant: The University of Birmingham, Birmingham (GB)

(72) Inventors: Felicity Jane de Cogan, Birmingham (GB); Graeme Forster, Durham (GB)

(73) Assignee: The University of Birmingham, Birmingham (GB)

(21) Appl. No.: 17/277,019

(22) PCT Filed: Sep. 20, 2019
(86) PCT No.: PCT/EP2019/075330

§ 371 Date: Mar. 17, 2021

(30) Foreign Application Priority Data
Sep. 20, 2018 (GB) 1815306.4

(51) Int. Cl. C23C 8/36; A61L 2/23; B05D 1/00; B05D 3/04; B05D 3/14

(52) U.S. Cl. CPC C23C 8/36; A61L 2/23; B05D 1/62; B05D 5/00; B05D 3/0486; B05D 3/148; B05D 3/0466

(57) ABSTRACT

A method of treating a polymer surface using conventional plasma nitriding, and a nitrided polymer surface obtained thereby. The method comprises introducing nitrogen into the polymer surface using conventional plasma nitriding, and optionally functionalizing the nitrided polymer surface with a molecule, such as an antimicrobial moiety, which is capable of forming a covalent bond with the nitrogen atoms within the polymer surface.

The present invention relates to a method of treating a polymer surface using conventional plasma nitriding, and a nitrided polymer surface obtained thereby.

Around one in every eleven patients acquires a nosocomial infection whilst in an NHS hospital at any given time. This costs the NHS hundreds of millions of pounds each year. Subsequently, reducing the spread of nosocomial infection is a key priority for the NHS.

Microbes have been shown to contaminate a multitude of surfaces, including, but not limited to, door handles, telephones, keyboards, taps, plastics and fabrics, such as scrubs and aprons. Further, it has been shown that 65% of nurses in direct contact with patients suffering from methicillin-resistant Staphylococcus aureus (MRSA) had contaminated clothing. An additional 42% of staff that had not been in direct contact with the infected patients, but had been in contact with surfaces in the hospital, also had contaminated clothing. This demonstrates that it is not only transmission between people that can enable a spread of infection, but also inanimate objects.

Following colonisation of a surface, a number of microbes have been shown to survive for a considerable amount of time. Some strains of MRSA have been seen to survive for up to 9 weeks after drying and parainfluenza virus has been shown to last up to 10 hours on a non-absorptive surface and 4 hours on an absorptive surface. These relatively long survival rates heighten the transmission capacity of the microbes in question by increasing the period of time in which they can infect other patients.

Despite the implementation of a hand washing protocol in UK hospitals, infection has still been able to spread. This may be due to varying compliance with the protocol itself or impracticality in the procedure, since staff come in to contact with a number of surfaces before they are able to reach a sink and wash their hands. This allows the contamination of surfaces from both staff and patients, enabling only a short period of relief from contamination for those who do wash their hands before they are re-infected. In addition, clothing can also become contaminated, providing an alternate route for the spread of microbes.

Although the cleaning of surfaces has been shown to decrease the spread of infection, it does not prevent re-colonization. Moreover, the initial cleaning process does not eradicate all microbes.

For example, MRSA is known to persist following cleaning. In addition, visible cleanliness does not necessarily correlate with the level of microbial contamination, making it difficult to assess if a surface has been cleaned correctly without doing further microbial tests. No standard for acceptable surface microbial load has been agreed and the infectious dose varies greatly between patients and studies making it difficult to assess how much and when cleaning must take place.

It is apparent that surfaces play an important role in the passage of infectious agents between individuals. Current cleaning processes are temporary and inefficient; improved long-term antimicrobial control is required. Not only is the spread of infectious agents important in a healthcare setting, but also in many other environments, including public buildings, business premises, domestic dwellings, public transport, farms and areas associated with food preparation.

WO2017/006139 describes metal surfaces functionalized with molecules comprising an antimicrobial moiety. The molecules were immobilized on the surfaces by bonding to nitrogen atoms within the metal surface which were introduced by a process known as plasma nitriding (also known as conventional plasma nitriding (DCPN) or ion nitriding). It was found that functionalizing a nitrided metal surface with an antimicrobial molecule is an efficient mechanism for controlling the spread of infection in areas which are difficult to clean, those that are not cleaned regularly, or those that are exposed to high numbers of pathogens. A nitrided metal surface functionalized with antimicrobial molecules such as peptides can advantageously be used to inhibit or reduce the growth of microorganisms on surfaces that are frequently touched, for example door handles.

Metal surfaces functionalized with antimicrobials in this way are therefore expected to be useful in helping to control infections, particularly in hospitals. The metal surfaces may be incorporated into articles such as worktops, chairs, tables, doors, handles, taps, railings and medical devices. The use of such surfaces is not limited to healthcare environments. Antimicrobial-functionalized metal surfaces may also be useful in public buildings, business premises, in a domestic dwelling, on farms or on public transport.

However, since many articles commonly used in hospitals are made from plastic, for example phones, keyboards and chairs, there remains a need for an improved mechanism for the antimicrobial control of the environment, particularly polymer surfaces.

Known methods for providing plastic articles with antimicrobial properties often involve adding antimicrobial moieties into the polymer material prior to moulding of components. This can have a detrimental effect on the polymer properties, and such additives can leach out during use of the articles, resulting in a surface that is no longer antimicrobial.

The present invention has been devised with these issues in mind.

According to a first aspect of the present invention, there is provided the use of conventional plasma nitriding for treating a polymer surface.

In a second aspect, the invention provides a method of treating a polymer surface, the method comprising introducing nitrogen into the polymer surface using conventional plasma nitriding. This results in the production of a nitrided polymer surface.

Conventional plasma nitriding uses a plasma discharge of reaction gases to heat a surface and to supply nitrogen ions. Nitriding is usually applied to metals such as iron, steel and titanium, as well as refractory materials, in order to increase hardness and improve resistance to corrosion. The conventional plasma nitriding process typically requires temperatures in the range of 350° C. to 600° C. Such temperatures are far in excess of what can be tolerated by polymer materials. It is therefore counter-intuitive that conventional plasma nitriding could be applied to polymers.

However, contrary to conventional wisdom, the present inventors have surprisingly found that polymer surfaces can be successfully nitrided using conventional plasma nitriding. By controlling the parameters of the process, the present inventors have found that it is possible to operate the conventional plasma nitriding process in a temperature range which is suitable for polymers.

The process of the invention uses a charged electron conductor gas (plasma) to give the reaction:

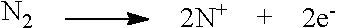

Under the temperatures at which the process is carried out, the absorption of 2N⁺ into the polymer surface is observed.

As will be appreciated by those skilled in the art, active screen plasma nitriding (ASPN) is an alternative technique to conventional plasma nitriding (DCPN). In ASPN, samples are enclosed by a screen, where a high cathodic potential is applied. The plasma acts on the screen rather than on the sample surface. Since the plasma is not formed directly on the sample surface, problems often associated with the DCPN process, such as edge effects, hollow cathode effects, non-uniformity of plasma temperature and arcing, are eliminated in ASPN. However, ASPN is a niche process which is not suitable for high-volume manufacturing. Further disadvantages of ASPN include high energy loss and difficulty of temperature control. The plasma parameters cannot be set independently of the nitriding temperature, since the plasma is the only energy source. Also, radicals are formed at the screen and are transported to the workpieces through a bias voltage. As the lifetime of these radicals is very short, only small laboratory furnaces can be used with uniform results. In contrast, DCPN is an energy-saving technology and provides excellent temperature uniformity. Since the ions are formed directly at the surface of the workpieces, uniform nitriding can be achieved. The plasma parameters can also be set independently of the nitriding temperature.

It will therefore be understood that the uses and methods of the present invention do not comprise ASPN.

The present inventors have developed a low energy plasma nitriding process that is suitable for nitriding polymers so as to embed nitrogen ions in the polymer surface. The nitriding process of the present invention limits the power density compared to the process normally used with metals.

As will be known by those skilled in the art, plasma nitriding is a gas nitriding process enhanced by a plasma discharge on the substrate to be nitrided (often referred to as the ‘workpiece’). The surface of the workpiece is enriched by nitrogen by a thermo-chemical treatment. The plasma is a gas that is ionized when exposed to an electrical potential. The substrate to be nitrided is typically placed in a vessel or furnace and is connected as a cathode. The vessel walls function as the anode. Particles are accelerated and hit the cathode (substrate), transferring all their kinetic energy and heating it. For gas particles to have sufficient kinetic energy most plasma nitriding processes are carried out under a partial vacuum.

Thus, in some embodiments the conventional plasma nitriding comprises:
placing an article comprising the polymer surface into a vessel; and
applying an electrical current between the polymer surface and a wall of the vessel in the presence of a gas comprising nitrogen.

The plasma is created by applying an electrical current between the cathodically charged workpiece (the polymer surface) and a wall of the vessel, which acts as the anode in the circuit. The plasma is ignited around the workpiece and activates the gaseous reactants that are supplied to the vessel.

The nitriding process is preferably carried out under a partial vacuum. In other words, the process may be carried out at a pressure which is lower than atmospheric pressure. The pressure of the gas inside the vessel may be no more than 9 mbar, no more than 8 mbar, no more than 7 mbar, no more than 6 mbar, no more than 5 mbar, no more than 4 mbar, no more than 3 mbar, or no more than 2.5 mbar. In some embodiments the pressure is at least 0.5 mbar, at least 1 mbar, at least 2 mbar or at least 3 mbar. For example, the pressure may be from 1 to 9 mbar, from 1.5 to 6 mbar, from 2 to 4 mbar or from 2.5 to 3.5 mbar. The pressure may be measured by a pressure sensor. The sensor may be located at the point of exit of the gas from the vessel, e.g. on a vacuum/suction line.

It will be appreciated that the gas used in the nitriding process must contain nitrogen. In some embodiments, the gas is a mixture of nitrogen and one or more other elements, such as argon or hydrogen. The gas may comprise at least 20%, at least 40%, at least 60% or at least 80% nitrogen (by volume). In some embodiments, the remainder is hydrogen. For example, a mixture of 25% nitrogen, 75% hydrogen may be used. In some embodiments a mixture of nitrogen, hydrogen and argon is used. It is preferred that high purity commercial grade gases are used.

The flow rate of the (or each) gas into the vessel may be from 1 to 150 litres/hour (l/h). The gas flow rate may be controlled by a mass flow controller (MFC), which regulates the flow to a set value. Each gas may be controlled by a separate MFC. The device may comprise a mass flow meter (MFM) and a programmable controller. The flow rate of argon, when present, may be from 1 to 50 l/h. The flow rate of nitrogen may be from 1 to 150 l/h. The flow rate of hydrogen may be from 1 to 150 l/h.

To avoid melting or degradation of the polymer, the temperature of the nitriding process must be controlled. In some embodiments, the temperature is no more than 100° C., no more than 90° C., no more than 80° C., no more than 70° C., no more than 60° C. or no more than 55° C. In some embodiments the temperature is from 40 to 90° C., from 50 to 80° C. or from 60 to 70° C. It will be appreciated that the exact temperature will depend on the polymer used and its melting point.

The vessel may be heated by one or more heaters. The heater(s) may be located outside of the vessel. Two, three, four or more heaters may be placed around the nitriding vessel. This helps to ensure an even temperature distribution and assist the plasma in heating the components.

The temperature may be measured using one or more thermocouples (e.g. Type K thermocouple). The thermocouples can be placed within the vessel to ensure that the temperature of the article(s) being nitrided is correctly monitored. Further thermocouples may also be fitted to various points within the vessel, for example on the fixtures, heat sinks and/base plate of the vessel. The thermocouples may be located on or near to the articles or surfaces to be treated. For example, the thermocouples may be attached to an internal frame within the vessel which holds the article being nitrided. It has been found that the frame temperature correlates well with that of the article being nitrided. Additionally or alternatively, thermocouples could be inserted into a test block manufactured from a similar material to that being nitrided.

The thermocouples can be set to a temperature selected by the operator. The thermocouples may be connected to a controller (for example a Programmable Logic Controller (PLC). The controller or system may be configured such that at 10-15° C. above the set point an alarm is activated and the nitriding process will abort.

The nitriding process may be carried out using any suitable equipment. Preferably the nitriding process of the invention is carried out using a plasma nitriding vacuum furnace. Plasma nitriding furnaces and systems are commercially available, as sold by Rubig, Henniker Plasma and B.M.I. For example, polymer surfaces may be nitrided using a Rubig Dual Station 150350 DC plasma nitriding unit. The nitrider may use an MPP-series or a MAP-series plasma generator. The Rubig system uses a Micropuls power supply, which enables the control of pulse cycles. This is particularly advantageous since it allows pulse patterns to be selected to suit the material and geometry of the article being nitrided.

The plasma is generated by applying a voltage between the polymer surface (which constitutes the cathodically charged workload) and the anodic vessel wall. In some embodiments, the voltage is from 250 V to 500 V, from 300 V to 375 V, or from 310 V to 350 V (e.g. 330 V).

Precise control of input voltage is critical to avoid exceeding the melting temperature of the polymer being processed. For example, for polycarbonate it is important not to exceed 65° C.

In some embodiments, a pulsed power pattern is used. The use of pulsed power means that the plasma current can be interrupted, rather than be a continuous current. This helps to ensure that arc discharging is eliminated and that a better control of heating can be achieved. For example, the power may be operated between a negative pulse and a positive pulse at a ratio of 3:1.

The pulse duration may be from 3 to 4000 μs, from 10 to 2000 μs, from 50 to 1000 μs, from 80 to 500 μs, from 100 to 250 μs or from 150 to 200 μs. The duration of each positive pulse may be the same as the duration of each negative pulse. For example, the duration of a pause between each pulse (i.e. the pulse pause) may be from 5 to 4000 μs, from 10 to 1000 μs, from 50 to 1000 μs, from 100 to 500 μs, or from 150 to 250 μs. In some embodiments the pulse pause may be longer than the pulse. In some embodiments the pulse pause is two times, three times, four times, five times, six times or seven times the pulse duration.

The following pulse settings may be used:
Only negative pulses: −1/+0;
Only positive pulses: −0/+1;
Alternating pulses: +1/−1; or
Bipolar pulsing from −2 to −250/+1. This achieves a better nitriding result inside holes and gaps.

In some embodiments the current is from 1 to 20 A, from 2 to 15 A or from 5 to 10 A.

The plasma nitriding process may be carried out for a period of from 30 minutes to 50 hours. In some embodiments, it is carried out for a period of from 1 hour to 25 hours, from 5 to 20 hours or from 10 to 15 hours.

In an exemplary embodiment, the vessel pressure is set at 3.5 mbar. The start voltage is set at 0 V, and the voltage finish set point is 320 V. The time taken to reach 320 V can be set at from 1000 to 9999 seconds, thereby controlling the ramp rate.

In some embodiments, the method further comprises a step of purging the vessel prior to carrying out the nitriding process. Purging may be carried out by reducing the pressure within the vessel. The pressure may be reduced to less than 5 mBar, less than 3 mBar or less than 2 mBar (e.g. to 1.5 mBar). The vessel may then be filled with nitrogen, optionally to a pressure of 20 to 100 mBar or 40 to 80 mBar (e.g. 50 mBar). The pressure may then be reduced again to less than 5 mBar, less than 3 mBar or less than 2 mBar (e.g. to 1.5 mBar). The pressure within the vessel may be reduced using a vacuum pump.

The polymer may be any suitable thermoplastic or thermosetting polymer. The polymer may comprise a synthetic or semi-synthetic organic compound. A skilled person will be capable of selecting a suitable polymer which has the desired properties for a particular application. For example, a polymer may be chosen that can be moulded into solid objects for use in applications where antimicrobial properties are required.

Suitable polymers include, but are not limited to: polyamide, polycarbonate, polyester, polyethylene (e.g. high-density polyethylene, low-density polyethylene, high molecular weight polyethylene, low molecular weight polyethylene), polyethylene terephthalate, polypropylene, polystyrene, polyurethanes, polyvinyl chloride, polyvinylidene chloride, acrylonitrile butadiene styrene, polyepoxide, polymethyl methacrylate, polytetrafluoroethylene, phenolics, melamine formaldehyde, urea formaldehyde, polyetheretherketone, maleimide, bismaleimide, polyetherimide, polyimide, plastarch material, polylactic acid, nylon, silicone, polysulfone, and combinations thereof. In some embodiments, the polymer is selected from the group consisting of polyvinyl chloride, melamine formaldehyde, polystyrene and polypropylene. In some embodiments, the polyvinyl chloride is unplasticised polyvinyl chloride (uPVC).

In some embodiments, the method further comprises functionalizing the nitrided polymer surface with a molecule which is capable of forming a covalent bond with the nitrogen atoms within the polymer surface.

The use of covalent interactions to immobilize molecules on a polymer surface is advantageous since covalent interactions strongly bind the molecules to the surface, thereby preventing or reducing their release. Molecules immobilized on a polymer surface by way of covalent interactions with nitrogen atoms within the surface are therefore expected to provide long-lasting benefits.

It will be appreciated that the molecule will comprise or consist of a functional group that is capable of forming a covalent bond with a nitrogen atom within the polymer surface. The functional group may be, but is not necessarily limited to, a carboxyl group, an amine, an imine, a thioamide or an enamine.

In some embodiments the molecule comprises a carboxyl group. It will be appreciated that in such embodiments, the covalent interaction is an amide bond formed between the carboxyl group of each molecule and a nitrogen atom in the polymer surface.

Thus, the use of a nitrided polymer surface enables the molecules to be bound directly to the surface, without the need for linkers or other functionalization of the polymer surface.

The molecules can be selected to provide any desired functionality. The attachment of molecules to surfaces to impart functionality is desirable in fields such as detection, for example, contamination detection or environmental monitoring. The ability to covalently immobilize antimicrobial molecules to a polymer surface is particularly useful in preventing the spread of infection.

Thus, in some embodiments the molecule comprises or consists of an antimicrobial moiety. The antimicrobial moiety may be antibacterial, antiviral and/or antifungal. The antimicrobial moiety may be an antibiotic, a biocide or a disinfectant (e.g. biguanide disinfectants, such as chlorhexidine).

In some embodiments the antimicrobial moiety is an antibiotic. The antimicrobial moiety may have a bacteriostatic or a bactericidal effect against Gram-positive bacteria, Gram-negative bacteria, or both. In some embodiments, the antimicrobial moiety thereof is effective against human or animal pathogens such as E. coli, S. aureus and P. aeruginosa.

The functional group which forms the covalent bond with the nitrided polymer surface may form a part of the antimicrobial moiety or it may be separate from the antimicrobial moiety. In embodiments wherein the functional group is part of the antimicrobial moiety, it will be understood that the antimicrobial moiety is directly bound to the polymer surface. In other words, there is no spacer between the polymer surface and the antimicrobial moiety.

In some embodiments, each molecule comprises a first antimicrobial moiety and a second moiety. It will be appreciated that the second moiety may have any desired function. The second moiety may also be antimicrobial, or it may be non-antimicrobial. For example, the second moiety may comprise one or more catalysis groups for the detection of contaminants, for example in fluid systems.

In some embodiments, the molecule comprises a spacer between the polymer surface and the (first and/or second) antimicrobial moiety. A spacer may advantageously allow the antimicrobial moiety freedom of movement and reduce steric hindrance when the molecule is bound to the surface. The spacer may have any desired size or length, for example, from 1 to 100 atoms, from 2 to 50 atoms or from 5 to 20 atoms.

In some embodiments the molecule or the antimicrobial moiety comprises or consists of a protein (e.g. pore forming toxin) or a peptide.

In some embodiments the molecule comprises, or is tagged with, a detectable label. Any suitable detectable label may be used. The detectable label may be, for example, a fluorescent label or a UV-visible label. Suitable fluorescent labels include FITC and rhodamine. A convenient UV-visible label is tryptophan, which can be incorporated into a peptide chain. The more tryptophan residues present in the chain, the stronger is the reading. Other means for incorporating detectable labels will be known to those skilled in the art. A detectable label provides a convenient means by which the presence of the molecules on a surface can be detected. This can be used for quality control, for example to verify that the molecules have been successfully immobilized on the surface. In addition, the presence of the immobilized molecules can be checked during the life of an article comprising the surface to indicate whether the surface retains its antimicrobial activity.

In some embodiments, the molecule, peptide or antimicrobial moiety has a minimum inhibitory concentration of no more than 10 mg/ml, no more than 5 mg/ml, no more than 2.5 mg/ml, no more than 1.25 mg/ml, no more than 1.0 mg/ml or no more than 0.5 mg/ml against E. coli, S. aureus and/or P. aeruginosa.

In some embodiments, the peptide is derived from a defensin.

Defensins are small peptides expressed by epithelial and immune cells, and display antimicrobial activity against many Gram-positive and Gram-negative bacteria, fungi and viruses. The defensin may be an alpha, a beta or a gamma defensin. By “derived from” it will be understood that the molecule, peptide or antimicrobial moiety may contain a part or the whole of the amino acid sequence of a defensin. Thus, in some embodiments, the peptide comprises or consists of a defensin peptide sequence, or a functional variant or fragment thereof.

The term “variant” of a defensin peptide sequence will be understood to mean that the peptide comprises or consists of a sequence having at least 30%, at least 40%, at least 50%, at least 60%, at least 70%, at least 80% or at least 90% identity with the amino acid sequence of a defensin peptide.

The term “fragment” will be understood to mean that the peptide comprises or consists of a portion of a defensin peptide. The fragment may be at least 30%, at least 40%, at least 50%, at least 60%, at least 70%, at least 80% or at least 90% of the whole of the defensin peptide. The fragment may include the C-terminus or the N-terminus of the defensin peptide, or it may include neither terminus.

By “functional”, it will be understood that the variant or fragment retains at least some of the antimicrobial activity of the defensin sequence from which it is derived. It will be appreciated that it may be possible to remove, add or replace one or more of the amino acids of a defensin peptide sequence to provide a variant or fragment which still displays antimicrobial activity. Indeed, a variant or fragment may have improved antimicrobial activity compared to its parent sequence.

The skilled technician will know how to produce fragments and variants of known defensin sequences and test their antimicrobial properties using standard techniques. The skilled technician will also know how to calculate the percentage identity between two amino acid sequences using well-known sequence alignment tools such as ClustalW (Thompson et al., 1994, Nucleic Acids Research, 22, 4673-4680; Thompson et al., 1997, Nucleic Acids Research, 24, 4876-4882).

The peptide may be from 5 to 40 amino acids in length. In some embodiments, the peptide has at least 6, at least 8 or at least 10 amino acids (residues). In some embodiments, the peptide has no more than 30, no more than 20, no more than 15 or no more than 12 amino acids.

The peptide may comprise at least 3, at least 4 or at least 5 amino acids having a positively charged side chain. The amino acids having a positively charged side chain may be arginine (R), histidine (H), lysine (K), or any combination thereof. The positively charged amino acids may be arranged consecutively, or they may be spaced apart from each other by one or more residues. Without wishing to be bound by theory, it is thought that the association of positive charges with a bacterial cell membrane may force pore formation and induce cell death.

In some embodiments, the peptide comprises at least 3 or at least 4 arginine residues. The arginine residues may be arranged consecutively, or they may be spaced apart from each other by one or more residues.

In some embodiments, the peptide comprises at least one sequence of 5 or more consecutive arginine residues.

In some embodiments, the peptide includes one or more hydrophobic and neutral amino acid residues. It is believed that the inclusion of hydrophobic and neutral residues confers broad spectrum activity. The peptide may include from 1 to 20, from 2 to 10 or from 3 to 6 hydrophobic amino acids. By “hydrophobic and neutral amino acid” we mean alanine (A), isoleucine (I), leucine (L), methionine (M), phenylalanine (F), tryptophan (W), tyrosine (Y), glycine (G), proline (P) or valine (V).

In some embodiments, the peptide comprises one or more hydrophilic amino acid residues. The peptide or antimicrobial moiety thereof may include from 1 to 20, from 2 to 10 or from 3 to 6 hydrophilic amino acids. By “hydrophilic amino acid” we mean serine (S), threonine (T), asparagine (N), glutamine (Q), aspartic acid (D), cysteine (C) or glutamic acid (E).

In some embodiments, the peptide comprises at least 3 positively charged amino acids and at least one hydrophobic amino acid and, optionally, at least one hydrophilic amino acid. In some further embodiments, at least two of the positively charged amino acids are separated from each other by one or more hydrophobic and/or hydrophilic residues.

In some embodiments, the peptide comprises or consists of a sequence having the formula A₍ₓ₎B_{(y)}A₍ₓ₎B_{(y)}, wherein:

A is a positively charged amino acid;

B is a hydrophobic amino acid or a hydrophilic amino acid;

x is a number of from 1 to 10; and

y is a number of from 1 to 10.

In some embodiments, the peptide comprises a sequence having the formula A₍ₓ₎Z_{(w)}B_{(y)}Z_{(w)}A₍ₓ₎Z_{(w)}B_{(y)}Z_{(w)}A₍ₓ₎ or the formula A₍ₓ₎B_{(y)}Z_{(w)}B_{(y)}A₍ₓ₎B_{(y)}Z_{(w)}B_{(y)}A₍ₓ₎ wherein:

A is a positively charged amino acid;

B is a hydrophobic amino acid;

C is a hydrophilic amino acid;

x is a number of from 1 to 6;

y is a number of from 1 to 6; and

z is a number of from 1 to 6.

In some embodiments, the peptide comprises or consists of the sequence RRYIGRGYIRR (SEQ ID No. 1).

In some embodiments, the peptide comprises or consists of the sequence RLYLRIGRR (SEQ ID No. 2).

In some embodiments, the peptide comprises or consists of the sequence CRVRGGRCA (SEQ ID No. 3).

In some embodiments, the peptide comprises or consists of the sequence RRRRRR (SEQ ID No. 4).

In some embodiments, the peptide comprises or consists of the sequence RRRRRRGALAGRRRRRRGALAG (SEQ ID No. 5).

In some embodiments, the peptide comprises or consists of the sequence GRRRRRRGALAGRRRRRRGALAG (SEQ ID No. 6).

In some embodiments, the peptide comprises or consists of the sequence KKKKKKGALAGKKKKKKGALAG (SEQ ID No. 7).

In some embodiments, the sequence of the peptide comprises a terminal cysteine residue. In further embodiments, the sequence of the peptide has a cysteine residue at each end. For example, the sequence RLYLRIGRR (SEQ ID No. 2) may be modified by the inclusion of terminal cysteine residues to give the sequence CRLYLRIGRRC (SEQ ID No. 8). In silico studies have suggested that the inclusion of cysteine residues may enable the peptide to reversibly cyclise through the formation of disulphide bridges, depending on the environment. Without wishing to be bound by theory, the present inventors hypothesise that the tertiary structure of the peptide may also influence its antimicrobial activity. Combining a three-dimensional structure with positive charges in the peptide may help to increase its efficacy.

Thus, in some embodiments, the peptide comprises or consists of a sequence having the formula CA₍ₓ₎B_{(y)}A₍ₓ₎B_{(y)}C, wherein:

A is a positively charged amino acid;

B is a hydrophobic amino acid or a hydrophilic amino acid;

x is a number of from 1 to 10;

y is a number of from 1 to 10; and

C is a cysteine residue.

In some embodiments, the peptide comprises or consists of the sequence CRLYLRIGRRC (SEQ ID No. 8), CRRRRRRGALAGRRRRRRGALAGC (SEQ ID No. 9), CGRRRRRRGALAGRRRRRRGALAGC (SEQ ID No. 10), CRVRGGRCAC (SEQ ID No. 11), CRRRRRRC (SEQ ID No. 12), CKKKKKKGALAGKKKKKKGALAGC (SEQ ID No. 13) or CRRYIGRGYIRRC (SEQ ID No. 14).

These sequences may be considered to be “synthetic defensins”, since they are similar in structure and function to naturally occurring defensins.

It will be understood that the term “a molecule”, as used herein, may refer to a plurality of molecules of a single type. Thus, the nitrided polymer surface may be functionalized with a plurality of molecules. In some embodiments, some or all of the molecules comprise or consist of an antimicrobial moiety.

In some embodiments, the method comprises functionalizing the nitrided polymer surface with a first molecule comprising an antimicrobial moiety, and a second molecule which is different to the first molecule. The second molecule may comprise an antimicrobial moiety. Alternatively, the second molecule may provide a different property.

In some embodiments the first and second molecules differ from each other by a single amino acid.

In some embodiments, the step of functionalizing the nitrided polymer surface with the molecules comprises contacting the nitrided polymer surface with the molecules, optionally in the presence of a catalyst.

It will be appreciated that the type of catalyst, if required, must be selected in accordance with the nature of molecules. The skilled person will be aware of suitable catalysts for coupling different functional groups to the nitrogen atoms in the polymer surface. In embodiments wherein the molecule is or comprises a peptide, a suitable catalyst is a mixture of HBTU ((2-(1H-benzotriazol-1-yl)-1,1,3,3-tetramethyluronium hexafluorophosphate)), and DIEA (N,N-Diisopropylethylamine).

The nitrided polymer surface may be contacted with the molecules by any suitable means. For example, the surface may be dip-coated or immersed in a solution containing the molecules and, optionally, a catalyst.

The nitrided polymer surface may be contacted with the molecules for a period of time suitable to enable covalent bonds to form between the molecules and the nitrogen atoms in the surface such that a desired density of molecules is achieved on the surface. In some embodiments, the surface is contacted with the molecules (e.g. a dipped or immersed in a solution containing the molecules) for a period of time of from 1 to 24 hours, e.g. approximately 12 hours.

In some embodiments, the method is carried out at a temperature of from 10 to 30° C., for example at room temperature.

It will be understood that all statements made herein in relation to the molecule may apply to the first molecule and/or to the second molecule.

According to a third aspect of the invention, there is provided a nitrided polymer surface.

The nitrided polymer surface may be producible by the method of the second aspect of the invention.

In some embodiments, the depth of the nitrogen in the polymer surface is at least 0.05 mm, at least 0.10 mm, at least 0.15 mm, at least 0.20 mm or at least 0.25 mm. In some embodiments, the depth of the nitrogen in the polymer surface is no more than 0.30 mm, no more than 0.25 mm or no more than 0.20 mm. The skilled person would be aware of techniques for measuring the depth of the nitrogen.

In some embodiments the surface is functionalized with a molecule comprising an antimicrobial moiety.

In some embodiments the invention provides a nitrided polymer surface functionalized with molecules, wherein all of the molecules are immobilized on the surface by only covalent interactions between the molecules and nitrogen atoms within the polymer surface.

The surface may be functionalised with two or more different molecules. In some embodiments the surface is functionalized with a first molecule comprising an antimicrobial moiety, and a second molecule which is different to the first molecule. For example, it may be advantageous to functionalize the surface with two or more different peptides comprising or consisting of antimicrobial moieties which are effective against different microorganisms. The skilled person may employ any number of combinations of molecules on the surface. Some or all of these molecules may bound by covalent attachments with nitrogen atoms within the polymer surface.

In some embodiments some or all of the molecules comprise or consist of peptides. In some embodiments, the peptides are immobilized on the surface by only covalent interactions between the C-terminal carboxyl groups of the peptides and nitrogen atoms within the polymer surface.

In some embodiments, there is provided a surface functionalized with at least one, at least two, at least three or at least five molecules, wherein each molecule differs from another. In some embodiments each molecule differs from another by chemical structure. In some embodiments each molecule differs from another by spectrum of activity. In some embodiments each molecule differs from another by mechanism of action. In some embodiments each molecule differs from another by class. The skilled person will be aware of the different classes of antimicrobials.

In some embodiments, there is provided a surface functionalized with at least one, at least two, at least three or at least five peptides, wherein each peptide differs from another by at least one amino acid.

The peptides may be formed by solid-phase peptide synthesis (SPPS). Further details with regards to SPPS will be known to those skilled in the art and can be found in common textbooks (e.g. Stryer, Biochemistry, W.H. Freeman and Co Ltd, 2002).

The functionalized polymer surface may be long-lasting. By long lasting, it will be understood that at least 50%, at least 60%, at least 70%, at least 80%, at least 90% or at least 95% of the molecules are immobilized on the surface for at least 1 week, at least 2, at least 3, at least 4, at least 5, at least 10 or at least 15 weeks.

The functionalized polymer surface may be hard-wearing. In some embodiments the molecules remain immobilized on the surface after mechanical and/or chemical treatment. The mechanical treatment may comprise regular contact with humans and/or animals. By “regular contact”, it will be understood that the surface is contacted at least once per day, at least once per week, at least once per month, at least once per 3 months or at least once per 6 months and that each contact lasts for at least one second. The chemical treatment may comprise treatment with alcohol (for example ethanol), Dettol®, trypsin, Vaseline®, detergent or soap, or any combination thereof. The surface may be contacted with the chemical treatment at least once per day, at least once per week, at least once per month, at least once per 3 months or at least once per 6 months and that each contact lasts for at least one second.

The nitrided polymer surface may be partially functionalized with molecules, i.e. only a portion of the polymer surface may be functionalized. Thus, in some embodiments, at least 40%, at least 50%, at least 60%, at least 70%, at least 80% or at least 90% of the area of the polymer surface has molecules immobilized thereon. In some embodiments, substantially the whole of the polymer surface has molecules immobilized thereon. The molecule percentage surface coverage of the metal may be determined by the use of fluorescent molecules and microscopy.

The density of molecules on the metal surface may be at least 50, at least 60, at least 70, at least 80 or at least 90 micromolar per cm² of surface.

The nitrided polymer surface may constitute a portion of or the whole of a surface of the article.

According to a further aspect of the invention, there is provided an article comprising the nitrided polymer surface of the third aspect of the invention. The nitrided polymer surface may constitute a portion of or the whole of a surface of the article.

The article may be, or may form a part of, a worktop, a chair, a desk or table, a door, a handle or a railing, a keypad or any other object that comes into regular contact with humans or animals. In particular, the article may be a tool or piece of apparatus used in healthcare or food preparation, such as a medical device. Applications for polymers having antimicrobial properties include healthcare, care homes, public transport, food production, farming, public buildings and sport facilities.

The medical device may be an implant (e.g. a dental implant, a pacemaker, a cochlear implant or an orthopaedic implant), a prosthesis (e.g. a prosthetic hip or knee, or a component thereof), analytical equipment, a catheter, or a surgical instrument.

In some embodiments, the nitrided polymer surface, or the article comprising said surface, is biocompatible. By “biocompatible”, it will be understood that the polymer surface is capable of existing within a human or animal body without having toxic or other deleterious effects on the human or animal. It is particularly preferred that the surface does not elicit an immune response.

It will be understood that all of the embodiments described herein may be combined in any way, and may apply to the first, second and third aspects of the invention as appropriate.

Embodiments of the invention will now be described by way of example and with reference to the accompanying Figures, in which:

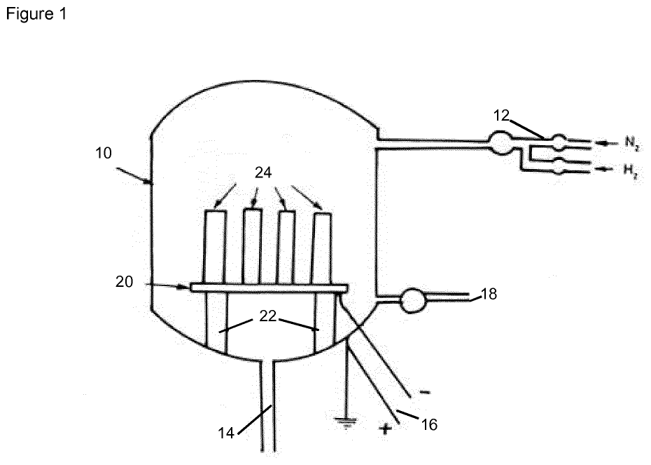

FIG. 1 is a schematic diagram of a nitriding vessel for use in a method of the present invention;

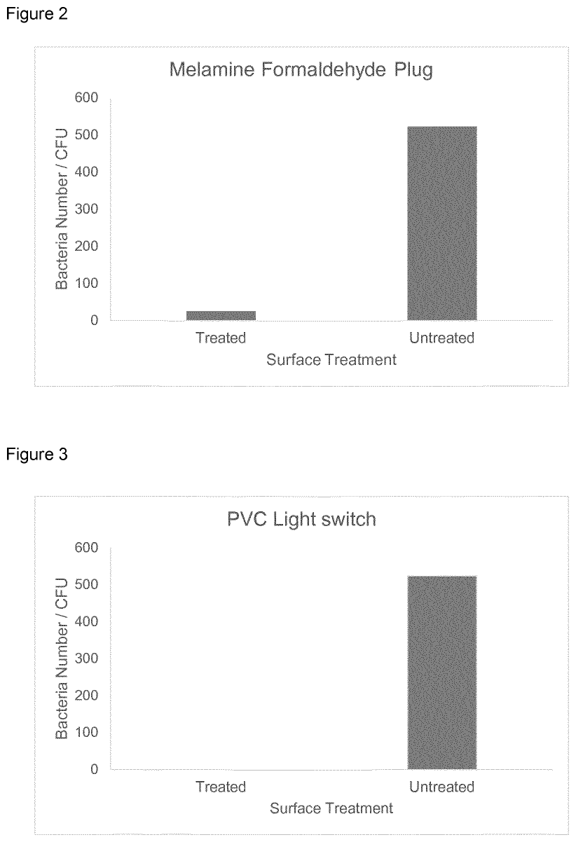

FIG. 2 is a graph showing the bacterial growth on a melamine formaldehyde plug which has been nitrided and functionalized with an antimicrobial, compared to an untreated plug;

FIG. 3 is a graph showing the bacterial growth on a PVC light switch which has been nitrided and functionalized with an antimicrobial, compared to an untreated light switch;

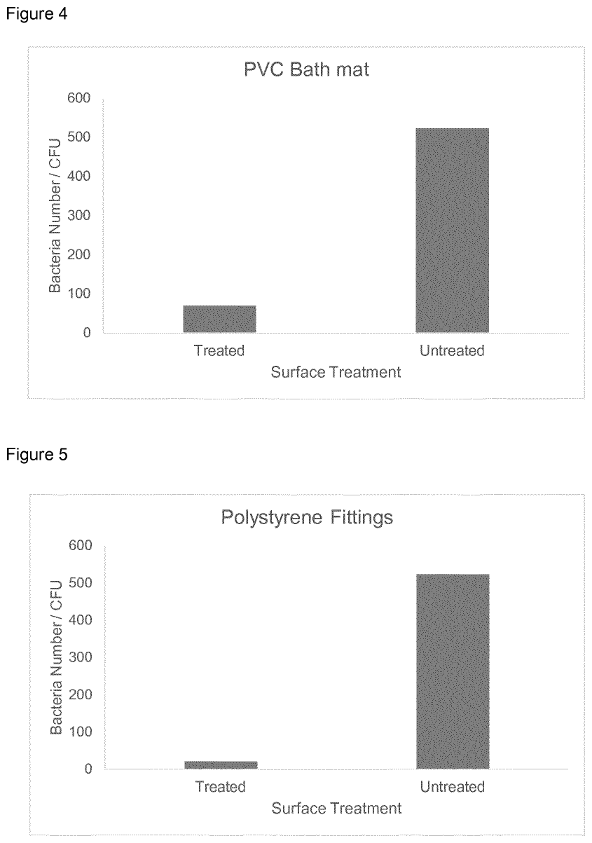

FIG. 4 is a graph showing the bacterial growth on a PVC bath mat which has been nitrided and functionalized with an antimicrobial, compared to an untreated bath mat;

FIG. 5 is a graph showing the bacterial growth on polystyrene fittings which has been nitrided and functionalized with an antimicrobial, compared to untreated fittings;

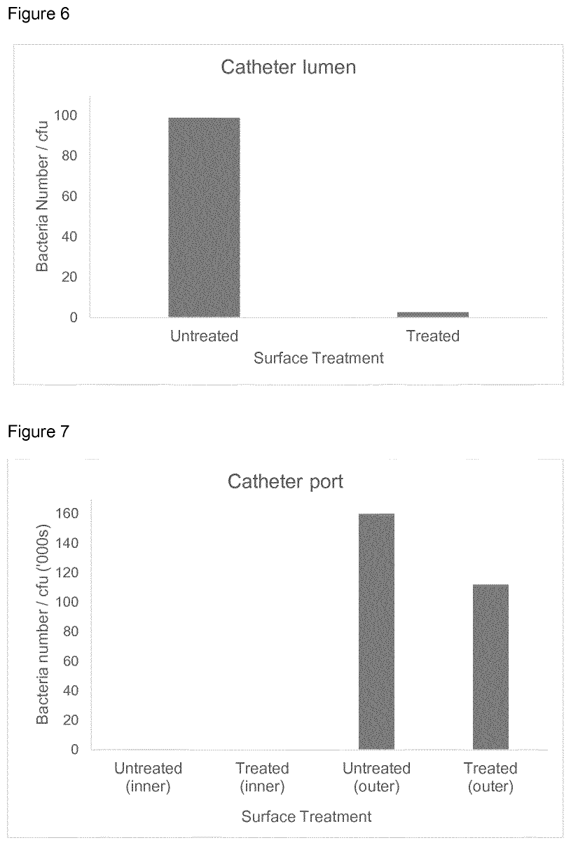

FIG. 6 is a graph showing the bacterial growth on the outer lumen of a silicone catheter which has been nitrided and functionalized with an antimicrobial, compared to an untreated silicone catheter;

FIG. 7 is a graph showing the bacterial growth on the port of a silicone catheter which has been nitrided and functionalized with an antimicrobial, compared to an untreated catheter port;

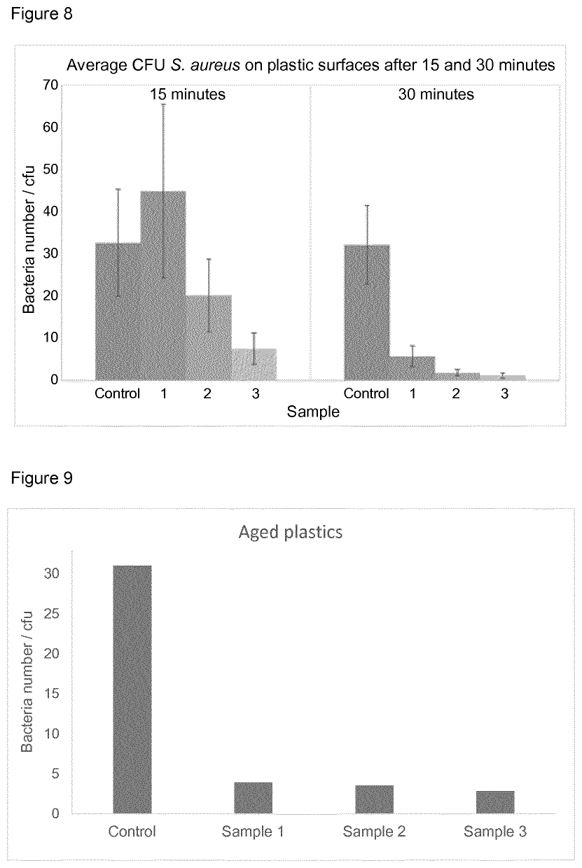

FIG. 8 is a graph showing the bacterial growth on plastic surfaces which have been nitrided and functionalized with an antimicrobial, compared to an untreated plastic surface, after 15 minutes and 30 minutes;

FIG. 9 is a graph showing the bacterial growth on aged plastic samples which have been nitrided and functionalized with an antimicrobial, compared to an untreated aged plastic sample; and

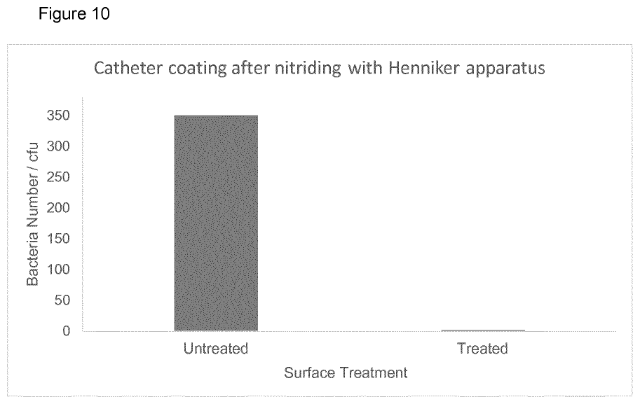

FIG. 10 is a graph showing the bacterial growth on a silicone catheter which has been nitrided using a Henniker apparatus and functionalized with an antimicrobial, compared to an untreated silicone catheter.

### EXAMPLE 1

Methodology

Nitriding

A polymer article (the workload) was loaded into a Rubig DC plasma nitriding vessel normally used for the treatment of steel and metal materials. A schematic diagram of a typical nitriding vessel 10 is shown in FIG. 1. The vessel 10 is connected to a gas supply 12, a vacuum pump 14, a power supply 16 and temperature controllers 18. Inside the vessel 10, a load plate 20 is positioned on supports 22. The load plate 20 receives the parts 24 (the work load) to be nitrided.

In the trials carried out, the work load was connected to the internal structure of the vessel by means of metal wire, although other materials could be used to locate the work load in the vessel. The pressure within the vessel was reduced using a vacuum pump to 1.5 mBar over a period of 5 minutes. The vessel was then backfilled with nitrogen (preferably high purity (99.9%) nitrogen) to a pressure of 50 mBar to purge the vessel before being pumped down again to a pressure of 1.5 mBar.

In a first stage external heaters were used to achieve a pre-set temperature value, prior to developing the plasma to create the ionisation phase. The vessel and the workload were heated to a temperature of 55° C.±10° C. using external wall heaters. The time taken to reach the pre-set temperature was typically in the range of 10 to 15 minutes. An atmosphere of 5% argon, 50% nitrogen, 45% hydrogen was used. Hydrogen is used in the mixture to remove any oxygen from the system prior to plasma nitriding. During this pre-heating phase the pressure is typically in the range of 100-200 mbar.

The vessel was pumped down again to 1.5 mBar to 2 mBar and then backfilled to a pressure of 2 mBar to 4 mBar with 75% H2:25% N₂. The pressure was maintained at the required level by a regulating valve on the vacuum pump.

A DC plasma voltage of 350V was applied between the cathodically charged polymer workload and the anodic vessel wall to generate the plasma in the gas mixture and embed the positively charged nitrogen ions in the polymer surface. For the purpose of the experimental runs the following parameters were employed:
Pulse duration (− and +) 100 μs
Pulse pause 150 μs.

The voltage was maintained for a period of 20 hours before the DC plasma voltage was turned off. The vessel was then backfilled with high purity nitrogen to atmospheric pressure so that the vessel could be opened to remove the workload.

Functionalizing Nitrided Surfaces with Antimicrobial Molecules

A variety of plastic articles were nitrided using the methods described above, including a melamine formaldehyde plug, a polyvinyl chloride (PVC) light switch, a PVC bath mat, polystyrene fittings, and a silicone catheter. The articles were functionalized by immersing them in a solution comprising acetonitrile (10 L), HBTU (100 g), DIEA (0.6 L) and chlorhexidine (0.1 L, 20% v/v stock solution) and agitated for 5 hours at room temperature before washing with water.

Testing of Antimicrobial Function

The nitrided functionalized surfaces were tested according to the ‘simulated splash model’, which compares coated and uncoated surfaces. An overnight subculture of MRSA in LB broth was prepared. The MRSA culture was applied to each surface in 1 uL drops in a grid pattern. The surfaces were incubated at room temperature for 10 minutes. The bacteria were then removed from the surface, plated out onto LB agar plates and incubated overnight at 37° C., and the colonies were counted. Each sample was tested in triplicate and the results averaged.

Results

FIGS. 2-7 show the effect of the nitrided functionalized surfaces on bacterial growth. As shown in FIG. 2, functionalizing the nitrided melamine formaldehyde plug with chlorhexidine resulted in a reduction in colony forming units (CFU) from over 500 to less than 30. Negligible bacterial growth was observed on the functionalized PVC light switch (FIG. 3). For the PVC bath mat, bacterial growth was reduced from over 500 to less than 100 CFUs (FIG. 4). Similarly, for the polystyrene fittings growth was reduced from over 500 to less than 30 CFUs (FIG. 5). For the silicone catheter, bacterial growth was reduced from 100 to less than 5 CFUs on the outer lumen (FIG. 6) and from 160 to less than 115 CFUs on the outside of the port (FIG. 7). These results show that a significant reduction in bacterial growth on polymer surfaces can be achieved by nitrided the surfaces and functionalizing the nitrided surface with an antimicrobial molecule.

### EXAMPLE 2

In a second example, plastic surfaces were nitrided and functionalized as described for Example 1 above. The plastic surfaces were tested with MRSA as described for Example 1 above, with one group of surfaces being incubated for 15 minutes and another group of surfaces being incubated for 30 minutes. FIG. 8 shows the effect of the nitrided functionalized surfaces on bacterial growth after 15 and 30 minutes, compared with a control sample. Sample 1 was a polypropylene surface, while samples 2 and 3 were uPVC surfaces. The graph in FIG. 8 shows that the bacterial growth was significantly reduced from over 30 CFUs to less than 10 CFUs after incubating for 30 minutes.

### EXAMPLE 3

In a third example, polypropylene and uPVC plastic pieces were nitrided and functionalized as described for Example 1 above and then aged to simulate 10 years of service. The aging was performed by tumbling the plastic pieces with zirconium oxide beads for 48 hours. The antimicrobial function was then tested with MRSA as described for Example 1 above, with the plastic pieces being incubated for 30 minutes.

FIG. 9 shows that, in the treated samples, bacterial growth was reduced from around 50 CFUs to less than 10 CFUs. This demonstrates the longevity of the antimicrobial effect that can be achieved by nitriding and functionalizing the nitrided surface with an antimicrobial molecule.

### EXAMPLE 4

In a fourth example, a silicone catheter was nitrided and functionalized as described for Example 1 above, but using a Henniker plasma nitriding unit instead of a Rubig plasma nitriding unit. The operating parameters of the Henniker apparatus were kept exactly the same as for the Rubig plasma nitriding unit. FIG. 10 shows that bacterial growth on the treated catheter was significantly reduced compared with an untreated catheter.

1: Use of conventional plasma nitriding for treating a polymer surface.

2: A method of treating a polymer surface, the method comprising introducing nitrogen into the polymer surface using conventional plasma nitriding, thereby producing a nitrided polymer surface.

3: The method of claim 2, wherein the conventional plasma nitriding comprises:
placing an article comprising the polymer surface into a vessel; and
applying an electrical current between the article and a wall of the vessel in the presence of a gas comprising nitrogen.

4: The method of claim 3, wherein the pressure of the gas inside the vessel is no more than 5 mbar.

5: The method of claim 3, wherein the gas comprises at least 20% nitrogen.

6: The method of claim 3, wherein the plasma nitriding process is carried out at a temperature of no more than 100° C.

7: The method of claim 3, wherein the plasma is generated by applying a voltage of from 300 V to 375 V between the polymer surface and the vessel wall.

8: The method of claim 3, wherein the current is from 5 to 10 A.

9: The method of claim 3, wherein the plasma nitriding process is carried out for a period of from 1 hour to 25 hours.

10: The method of claim 3, further comprising a step of purging the vessel prior to carrying out the nitriding process.

11: The method of claim 2, wherein the polymer is polyamide, polycarbonate, polyester, polyethylene, polyethylene terephthalate, polypropylene, polystyrene, polyurethane, polyvinyl chloride, polyvinylidene chloride, acrylonitrile butadiene styrene, polyepoxide, polymethyl methacrylate, polytetrafluoroethylene, phenolics, melamine formaldehyde, urea formaldehyde, polyetheretherketone, maleimide, bismaleimide, polyetherimide, polyimide, plastarch material, polylactic acid, nylon, silicone, polysulfone, or a combination thereof.

12: The method of claim 2, further comprising the step of functionalizing the nitrided polymer surface with a molecule which is capable of forming a covalent bond with the nitrogen atoms within the polymer surface.

13: The method of claim 12, wherein the molecule comprises an antimicrobial moiety.

14: The method of claim 13, wherein the antimicrobial moiety is antibacterial.

15: The method of claim 13, wherein the molecule or the antimicrobial moiety is a peptide, optionally wherein the peptide comprises or consists of a defensin peptide sequence, or a fragment or variant thereof.

16: A nitrided polymer surface producible by the method of claim 1.

17: A nitrided polymer surface, wherein the surface is functionalized with a molecule comprising an antimicrobial moiety.

18: The nitrided polymer surface of claim 17, wherein the surface is functionalized with a first molecule comprising an antimicrobial moiety, and a second molecule which is different to the first molecule.

19: An article comprising the nitrided polymer surface of claim 16, wherein the nitrided polymer surface constitutes a portion of or the whole of a surface of the article.
